(19) United States
(12) Patent Application Publication
(10) Pub. No.: US 2006/0163100 A1
(43) Pub. Date: Jul. 27, 2006

(54) Containers sealed with flexible film with means for preventing seal failures caused by inner pressure increase

(76) Inventor: Eric Jonsen, Seattle, WA (US)

Correspondence Address: PHILIPS INTELLECTUAL PROPERTY & STANDARDS, P.O. BOX 3001, BRIARCLIFF MANOR, NY, 10510, US

(21) Appl. No.: 10/537,795

(22) PCT Filed: Dec. 10, 2003
(86) PCT No.: PCT/IB03/05901

§ 371 Date: Mar. 13, 2006

Related U.S. Application Data
(60) Provisional application No. 60/434,589, filed on Dec. 18, 2002.

(51) Int. Cl. A61B 19/02

(52) U.S. Cl. 206/438

(57) ABSTRACT

A sealed container includes a tub (14), a lid (11), a flexible seal (15) disposed between the tub and the lid, and one or more ribs (12a-12b) disposed on an interior side of the lid (11) that place pressure on the flexible seal (15) at potential seal failure points when the lid (11) is attached. The one or more ribs provide additional force to the flexible seal at places on the flexible seal where peeling of the seal is most likely to originate. The function of the rib is to restrain the film under pressurized conditions, but under normal conditions the rib may not contact the flexible seal.

The present invention is directed to methods and apparatuses for storing items, and more particularly to a method and apparatus for storing items in a sealed container that is sealed with a flexible film.

The current state of the art in film sealed packages includes either flexible packaging (e.g., bags, pouches, and thermoformed pill packaging) or relatively rigid containers, such as pill bottles, food containers, and packaging for medical disposables, with flexible film bonded over the opening of the container. Under certain conditions, pressure builds up inside the sealed container placing strain on the seal. This leads to seal failures, which can cause spoilage, dehydration and other problems.

The present invention is therefore directed to the problem of developing a method and apparatus for storing items in a sealed container that is sealed with a flexible seal but yet can withstand pressure changes without causing seal failures.

The present invention solves these and other problems by providing inter alia a sealed container including a tub, a lid, a flexible seal disposed between the tub and the lid, and one or more ribs disposed on an interior side of the lid that place pressure on the flexible seal at potential seal failure points when the lid is attached. The one or more ribs provide additional force to the flexible seal at places on the flexible seal where peeling of the seal is most likely to originate. The function of the rib is to restrain the film under pressurized conditions, but under normal conditions the rib may not contact the flexible seal.

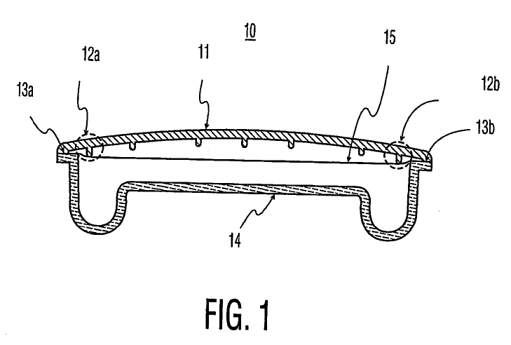

FIG. 1 depicts an exemplary embodiment of an apparatus for storing items in a sealed, rigid container according to one aspect of the present invention.

It is worthy to note that any reference herein to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment.

The present invention herein is applicable to any container comprised of a rigid shell sealed with flexible film that is protected by a rigid lid. One embodiment of the present invention includes a one or more continuous ribs disposed in the container lid. At least one rib is positioned just inside the seal zone and extends slightly below the plane of the seal joint. This rib prevents the seal from rising above the plane of the seal joint and initiating a peel failure when there is positive pressure inside the container due to changes in ambient conditions. Additional ribs may be included to provide additional force against the seal at several points along the length of the seal.

The present invention adds ruggedness to rigid containers that have been sealed using seal materials and processes that are readily available in the industry. Specifically, this design allows the seal opening to be larger and/or broadens the environmental range in which the package can be used.

The size of the opening to be sealed is important in the case of a rigid container because any positive internal pressure results in force acting against the seal film. This force is equal to the area of the film, multiplied by the internal pressure. When the sealed container is subject to ambient conditions that result in a pressure increase inside the container, the film begins to be stretched by the expanding air trapped inside. At some critical point, the film hits its expansion limit and the seal joint between the rigid container and the film begins to experience a loading condition that if not stopped will begin to peel the joint apart from the inside out. The rib design herein prevents that mode of failure and allows this sealing method to be used where previously the sealed opening would have been larger than the foreseeable operating environment would allow. This is particularly important for larger items that can benefit from the cost savings of this packaging method but must preserve specific attributes such as sterility or moisture content, which require an intact seal.

Turning to FIG. 1, shown therein is an exemplary embodiment 10 of an apparatus for storing items in a sealed environment. The embodiment 10 is applicable to any sealed container comprised of a rigid shell 14 sealed with a flexible film 15 that is protected by a rigid lid 11. It consists of a continuous rib 12a, 12b attached to the lid 11 that protrudes slightly below the plane of the seal joint and is positioned just inside the seal perimeter or seal zone 13a, 13b. The lid 11 is firmly attached to the shell 14. This may be achieved by, but is not limited to, multiple part latching mechanisms, mating threads, or bayonet type latches. The rib 12a, 12b functions by preventing the seal film 15 from rising above the plane of the seal joint when a positive pressure exists within the container. By restraining the film 15 only near the seal joint the rib 12a, 12b prevents the peel modes of failure described above, while allowing the remaining film surface to expand and help absorb changes in the internal pressure. It should also be noted that a complete lid is not required for this invention to work. A partial lid with an open area inside the restraining rib will also be effective.

According to some embodiments of the present invention, the rib can run along the inside of the perimeter of the rim surface. Thus, if the rim surface has a perimeter shaped as a circle or an ellipse, the rib also forms a circle or an ellipse, respectively, having a diameter slightly less than the diameter of the circle formed by the rim surface. Also, for example, if the rim surface has a perimeter shaped as a square, the rib also forms a square, having a side length slightly less than the side length of the square formed by the rim surface.

Thus, there are at least two possible implementations of the rib. In one implementation, the rib substantially follows the path of the rim surface. In other possible implementations, the rib does not follow the path of the rim surface. In a preferred embodiment, a contiguous rib runs just to the inside of the seal rim. This design maximizes the rib's ability to hold the seal film at or below the plane of the rim while minimizing the force required to accomplish this. However, in other possible alternatives the rib does not exactly follow the path of the rim. For example, if the rim formed a perfect square opening, the rib could have radii at its corners and be slightly rectangular. Yet other possibilities include non-contiguous ribs, or staggered ribs.

If the above container is used to store electrode pads for use with an automatic external defibrillator, these pads are disposed inside the tub in a controlled atmosphere. Other uses for the above container are also possible, such as for use in storing medicine, pills, food, medical disposables, etc.

Although various embodiments are specifically illustrated and described herein, it will be appreciated that modifications and variations of the invention are covered by the above teachings and are within the purview of the appended claims without departing from the spirit and intended scope of the invention. For example, the description above relates to a container for storing defibrillator pads; however, other possible uses are also envisioned, such as food, or medical supplies. Furthermore, these examples should not be interpreted to limit the modifications and variations of the invention covered by the claims but are merely illustrative of possible variations.

1. A storage container comprising:
a rigid tub (14) having a rim surface;
a rigid lid (11);
a flexible seal film (15) forming a seal between the tub (14) and the lid (11) on the rim surface; and
one or more restraining ribs (12a-12b) disposed on the lid (11) and jutting out from the lid (11) towards the tub (14), at least one of said one or more restraining ribs (12a) to press against the seal film (15) as the seal film (15) expands due to changes in ambient conditions.

2. The container according to claim 1, wherein at least one of the one or more ribs (12a) applies pressure against the seal film (15) along a curve just inside a perimeter of the seal film (15).

3. The container according to claim 1, wherein at least one of the one or more ribs (12a) applies pressure against the seal film (15) to maintain some portion of the seal film (15) at or below a plane formed by the rim surface.

4. The container according to claim 1, wherein the one or more ribs (12a) comprise at least two ribs (12a-b), and the at least two ribs (12a-b) apply pressure against the seal film (15) along two lines just before where the seal film (15) meets the rim surface.

5. The container according to claim 1, wherein the one or more ribs (12a) comprise at least two ribs (12a-b), and at least two ribs (12a-b) apply pressure against the seal film (15) to maintain some portion of the seal film (15) at or below a plane formed by the rim surface.

6. The container according to claim 1, wherein the one or more ribs (12a) run continuous along a length of the lid (11).

7. The container according to claim 1, wherein each of the one or more ribs (12a) forms a closed curve substantially similar in shape to a curve formed by the rim surface.

8. The container according to claim 1, wherein each of the one or more ribs (12a) forms a non-contiguous curve.

9. The container according to claim 1, wherein each of the one or more ribs (12a) comprises a staggered set of ribs.

10. The container according to claim 1, further comprising electrode pads for use with an automatic external defibrillator disposed inside the tub (14).

11. The container according to claim 1, wherein the container is used to store one or more of the following: medicine, pills, food, and medical disposables.

12. The container according to claim 1, wherein the lid (11) is firmly attachable to the tub (14).

13. An apparatus for storing electrode pads for an automatic external defibrillator comprising:
a container (14) having a rim surface;
a lid (11);
a flexible seal film (15) forming a seal between the container (14) and the lid (11) on the rim surface; and
at least one continuous rib (12a) disposed on the lid (11) and extending out from the lid (11) towards the container (14) and below a plane of the rim surface, said at least one continuous rib (12a) to press against the seal film as the seal film expands due to changes in ambient conditions.

14. The apparatus according to claim 13, wherein said at least one rib (12a) applies pressure against the film (15) along a curve just inside a perimeter of the seal film (15).

15. The apparatus according to claim 13, wherein said at least one rib (12a) applies pressure against the film (15) to press the film (15) below the plane formed by the rim surface.

16. The apparatus according to claim 13, further comprising at least two ribs (12a-b), wherein the at least two ribs (12a-b) apply pressure against the seal film (15) along two lines just before where the film (15) attaches to the rim surface.

17. The apparatus according to claim 13, further comprising at least two ribs (12a-b), wherein the at least two ribs (12a-b) apply pressure against the seal film (15) to press the seal film (15) below a plane formed by the rim surface.

18. The apparatus according to claim 13, wherein the at least one rib (12a) runs continuous along a length of the lid (11).

19. The apparatus according to claim 13, wherein each of the one or more ribs (12a) forms a closed curve substantially similar in shape to a curve formed by the rim surface.

20. The apparatus according to claim 13, wherein each of the one or more ribs (12a) forms a non-contiguous curve.

21. The apparatus according to claim 13, wherein each of the one or more ribs (12a) comprises a staggered set of ribs.

22. The apparatus according to claim 13, further comprising electrode pads for use with an automatic external defibrillator disposed inside the tub (14).

23. An apparatus comprising:
a sealed tray (14) including a film (15) forming a seal over the tray (14); and
a lid (11) to be placed on the sealed tray (14), said lid (11) including one or more restraining ribs (12a) jutting out from one side of the lid (11), at least one of said one or more restraining ribs (12a) to press against the film (15) as the film (15) expands due to changes in ambient conditions.
